(19) United States
(12) Patent Application Publication
(10) Pub. No.: US 2022/0121659 A1
(43) Pub. Date: Apr. 21, 2022

(54) SYSTEM AND METHOD THAT FACILITATES MATCHING PETS WITH PROSPECTIVE ADOPTERS

(71) Applicant: Diana Diaz, Visalia, CA (US)

(72) Inventor: Diana Diaz, Visalia, CA (US)

(21) Appl. No.: 17/504,422

(22) Filed: Oct. 18, 2021

Related U.S. Application Data
(60) Provisional application No. 63/093,067, filed on Oct. 16, 2020.

(51) Int. Cl. G06F 16/248; G06F 16/2457; G06F 9/451; G16H 10/60

(52) U.S. Cl. CPC G06F 16/248; G16H 10/60; G06F 9/451; G06F 16/24575

(57) ABSTRACT

Aspects directed towards matching pets with prospective adopters are disclosed. In a particular aspect, a plurality of profiles are generated which include a plurality of pet owner profiles, a plurality of pet adopter profiles, and a plurality of pet profiles. At least a first one of the plurality of profiles is matched with at least a second one of the plurality of profiles based on criteria included the first one of the plurality of profiles and the second one of the plurality of profiles. An escrow process is then facilitated between a first user corresponding to the first one of the plurality of profiles and a second user corresponding to the second one of the plurality of profiles.

### TECHNICAL FIELD

This application claims the benefit of U.S. Provisional Patent Application Ser. No. 63/093,067, filed Oct. 16, 2020, which is titled “SYSTEM AND METHOD THAT FACILITATES MATCHING PETS WITH PROSPECTIVE ADOPTERS” and its entire contents of which are incorporated herein by reference.

### TECHNICAL FIELD

The subject disclosure generally relates to pets, and more specifically to matching pets with prospective adopters.

### BACKGROUND

By way of background concerning conventional pet adoption methods, it is noted that pet owners wanting to put their pet up for adoption typically go to an animal shelter, which is often undesirable. For instance, in addition to having to travel to an animal shelter, a pet owner may be reluctant to leave their pet at an animal shelter where they might not know how their pet will be treated and/or the conditions of their pet's future home. Alternatively, a pet owner might simply post that their pet is up for adoption online, but current sites lack a mechanism for vetting candidate adopters ahead of time.

Accordingly, it would be desirable to provide a system and method which overcomes these limitations. To this end, it should be noted that the above-described deficiencies are merely intended to provide an overview of some of the problems of conventional systems, and are not intended to be exhaustive. Other problems with the state of the art and corresponding benefits of some of the various non-limiting embodiments may become further apparent upon review of the following detailed description.

### SUMMARY

A simplified summary is provided herein to help enable a basic or general understanding of various aspects of exemplary, non-limiting embodiments that follow in the more detailed description and the accompanying drawings. This summary is not intended, however, as an extensive or exhaustive overview. Instead, the sole purpose of this summary is to present some concepts related to some exemplary non-limiting embodiments in a simplified form as a prelude to the more detailed description of the various embodiments that follow.

In accordance with one or more embodiments and corresponding disclosure, various non-limiting aspects are described in connection with matching pets with prospective adopters. In one such aspect, a method is disclosed which includes generating a plurality of profiles such as a plurality of pet owner profiles, a plurality of pet adopter profiles, and a plurality of pet profiles. The method further includes matching at least a first one of the plurality of profiles with at least a second one of the plurality of profiles based on criteria included the first one of the plurality of profiles and the second one of the plurality of profiles. An escrow process is then facilitated between a first user corresponding to the first one of the plurality of profiles and a second user corresponding to the second one of the plurality of profiles.

In another aspect, a system is provided. Within such embodiment, the system includes a communication component, a match component, and an escrow component. The communication component is configured to generate a plurality of profiles such as a plurality of pet owner profiles, a plurality of pet adopter profiles, and a plurality of pet profiles. The match component is configured to match at least a first one of the plurality of profiles with at least a second one of the plurality of profiles based on criteria included the first one of the plurality of profiles and the second one of the plurality of profiles. The escrow component is then configured to facilitate an escrow process between a first user corresponding to the first one of the plurality of profiles and a second user corresponding to the second one of the plurality of profiles.

In yet another aspect, a computer-readable storage medium is provided. The computer-readable storage medium comprises a memory component configured to store computer-readable instructions that include instructions for performing various acts. For this particular embodiment, the acts include generating a plurality of profiles such as a plurality of pet owner profiles, a plurality of pet adopter profiles, and a plurality of pet profiles. The acts further include matching at least a first one of the plurality of profiles with at least a second one of the plurality of profiles based on criteria included the first one of the plurality of profiles and the second one of the plurality of profiles. An escrow process is then facilitated between a first user corresponding to the first one of the plurality of profiles and a second user corresponding to the second one of the plurality of profiles.

Other embodiments and various non-limiting examples, scenarios and implementations are described in more detail below.

### BRIEF DESCRIPTION OF THE DRAWINGS

Various non-limiting embodiments are further described with reference to the accompanying drawings in which:

FIG. 1 illustrates exemplary user interfaces for matching pets with prospective adopters in accordance with an aspect of the subject specification;

FIG. 2 illustrates an exemplary environment that facilitates matching pets with prospective adopters in accordance with an aspect of the subject specification;

FIG. 3 is a block diagram illustrating exemplary components of a management system in accordance with an aspect of the subject specification;

FIG. 4 is a flow diagram of an exemplary methodology that facilitates matching pets with prospective adopters in accordance with an aspect of the subject specification;

FIG. 5 is a block diagram representing exemplary non-limiting networked environments in which various embodiments described herein can be implemented; and

FIG. 6 is a block diagram representing an exemplary non-limiting computing system or operating environment in which one or more aspects of various embodiments described herein can be implemented.

### DETAILED DESCRIPTION

### Overview

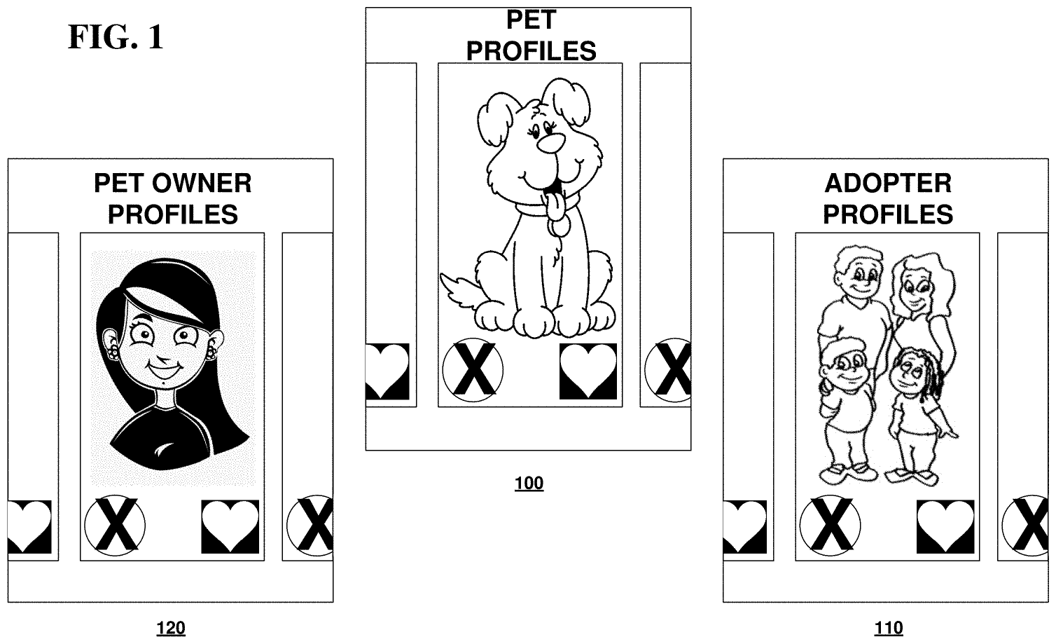

As discussed in the background, it is desirable to provide a system and method which overcomes the various limitations of conventional ways in which pets are matched with prospective adopters, wherein it should be appreciated that prospective adopters are broadly defined to be either permanent or temporary (e.g., where temporary pet-sitting by the pet owner is desired). The embodiments disclosed herein are directed towards overcoming such limitations by providing a digital pet-matching platform in which pet owners and prospective pet adopters can readily vet each other. In a particular embodiment, a computer application is contemplated (e.g., mobile or web-based) in which pet owners and prospective pet adopters may pre-screen each other prior to connecting according to their respective preferences. For instance, as illustrated in FIG. 1, a user interface for a prospective pet adopter is contemplated in which a series of pet profiles 100 and/or pet owner profiles 120 are displayed that meet the user's search criteria (e.g., dogs of a particular breed, below a particular age, within a desired geographic radius, etc.; or owners having a particular socioeconomic background, living within a desired geographic radius, etc.). The prospective pet adopter may then swipe left/right to browse through the series of pets and click on a desired profile to learn more details. Similarly, a user interface is contemplated for pet owners to browse through a series of prospective pet adopter profiles 110 that meet the user's search criteria (e.g., prospective adopters with a backyard, children, and other dogs). The pet owner may then swipe left/right to browse through the series of prospective adopters and click on a desired profile to learn more details.

Aspects disclosed herein also include a digital escrow process to facilitate the re-homing of a pet once a preliminary match is made between a pet owner and a prospective pet adopter. Such a process may, for example, include payment of a re-homing fee from the prospective pet adopter to open escrow. As part of the escrow process, it is further contemplated that a data room may be configured to receive any of various due diligence items from either the prospective pet adopter (e.g., video of backyard) or the pet owner (e.g., proof of vaccinations). Once all the conditions of escrow are met, it is contemplated that the system disclosed herein can facilitate a transfer of funds, if any, from the prospective pet adopter to the pet owner and/or vice versa.

It should be appreciated that conventional match-making sites exist, such as the match-making aspects disclosed in U.S. Pat. No. 9,733,811, which is hereby incorporated by reference in its entirety. Unlike conventional match-making sites, however, the aspects disclosed herein are directed towards matching pets with prospective adopters, rather than matching people with other people. Accordingly, various pet-specific aspects are contemplated herein that are unique to the match-making space including, but not limited to, aspects directed towards leveraging external resources to facilitate matches and/or to confirm any of various escrow conditions.

### Exemplary Pet Matching Environment

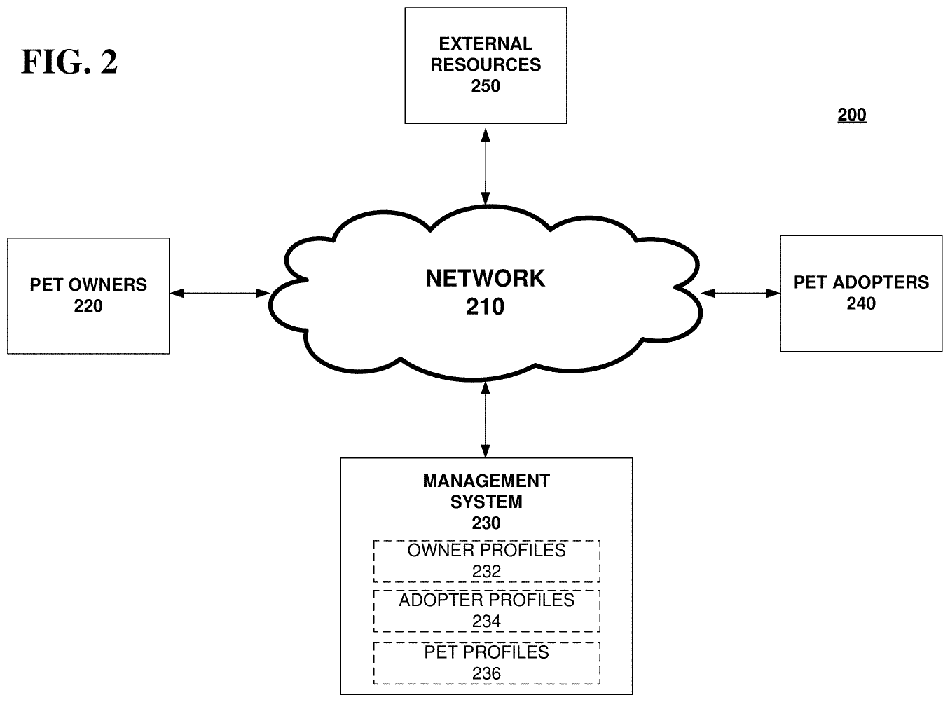

Referring next to FIG. 2, an exemplary environment that facilitates matching pets with prospective adopters in accordance with an aspect of the subject specification is provided. As illustrated, environment 200 includes pet owners 220, pet adopters 240, and external resources 250, which are communicatively coupled to a management system 230 via a network 210 (e.g., the Internet). Within such embodiment, it is contemplated that the management system 230 may be configured to facilitate the creation and storage of various profiles including, for example, owner profiles 232, adopter profiles 234, and pet profiles 236, as shown. It is also contemplated that the management system 230 may be further configured to receive search criteria from pet owners 220 and/or pet adopters 240 to generate corresponding search results, which are then displayed to the pet owners 220 and/or pet adopters 240 on their respective devices (e.g., smartphones configured to provide user interfaces 100 and 110, as illustrated in FIG. 1).

Here, it should be appreciated that external resources 250 may be used in any of various ways. For instance, pet owners 220 and/or pet adopters 240 may be connected to external resources 250 during the match process. For instance, management system 230 may be configured to provide pet owners 220 and/or pet adopters 240 with links to any of various external resources 250 associated with attributes included in any of owner profiles 232 (e.g., links to social media accounts associated with owner), adopter profiles 234 (e.g. links to social media accounts associated the adopter), and/or pet profiles 236 (e.g., links to websites providing details about a particular breed). Management system 230 may also be configured to facilitate escrow via any of various external resources 250 to vet attributes associated with any of owner profiles 232 (e.g., confirming whether owner has a criminal record), adopter profiles 234 (e.g. confirming details associated with listed address of the adopter via real estate websites; confirming whether adopter has history of animal cruelty), and/or pet profiles 236 (e.g., confirming that pet has all vaccinations via accredited animal hospital).

### Exemplary Management System

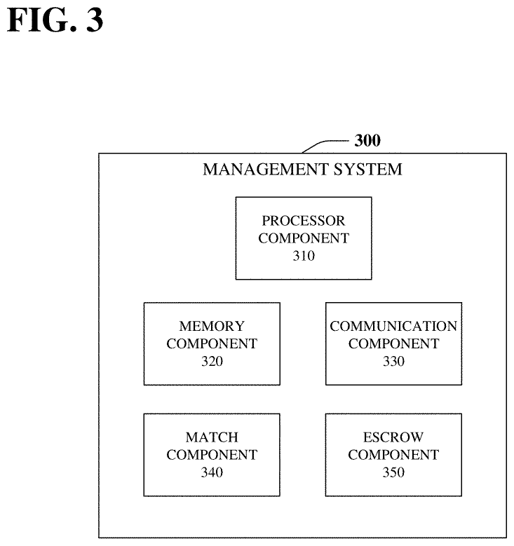

FIG. 3 shows a block diagram of an exemplary management system 300 which facilitates various aspects disclosed herein. As shown in FIG. 3, management system 300 may include a processor component 310, a memory component 320, a communication component 330, a match component 340, and an escrow component 350. Components 310-350 may reside together in a single location or separately in different locations in various combinations, including, for example, a configuration in which any of the aforementioned components reside in a cloud. For instance, with reference to FIG. 1, it is contemplated that these components may reside, alone or in combination, in computing devices corresponding to any of pet owners 220, management system 230, and/or pet adopters 240.

In one aspect, processor component 310 is configured to execute computer-readable instructions related to performing any of a plurality of functions. Processor component 310 can be a single processor or a plurality of processors which analyze and/or generate information utilized by memory component 320, communication component 330, match component 340, and/or escrow component 350. Additionally, or alternatively, processor component 310 may be configured to control one or more components of management system 300.

In another aspect, memory component 320 is coupled to processor component 310 and configured to store computer-readable instructions executed by processor component 310. Memory component 320 may also be configured to store any of a plurality of other types of data including data generated by any of communication component 330, match component 340, and/or escrow component 350. Memory component 320 can be configured in a number of different configurations, including as random access memory, battery-backed memory, Solid State memory, hard disk, magnetic tape, etc. Various features can also be implemented upon memory component 320, such as compression and automatic back up (e.g., use of a Redundant Array of Independent Drives configuration). In one aspect, the memory may be located on a network, such as a “cloud storage” solution.

As illustrated, management system 300 may also comprise communication component 330, which is configured to interface management system 300 with pet owners and prospective pet adopters (e.g., when collecting profile data, search criteria, etc.), as well as to facilitate communications between pet owners and prospective pet adopters when there is a preliminary match.

As illustrated in FIG. 3, management system 300 may also comprise match component 340, which is configured to match pet owners and prospective pet adopters according to their respective match criteria. For instance, match component 340 may include digital logic configured to determine which pet profiles (e.g., pet profiles 236) match a prospective pet adopter's search criteria, as well as which prospective pet adopter profiles (e.g., adopter profiles 234) match a pet owner's search criteria. Match component 330 may then be further configured to generate a series of profiles that meet the user's search criteria, which are then transmitted to the user via communication component 330.

Management system 300 may also comprise escrow component 350, which is configured to facilitate a digital escrow process once a preliminary match is made between a pet owner and a prospective pet adopter. Escrow component 350 may, for example, be configured to facilitate payment of a re-homing fee from the prospective pet adopter to open escrow. It is further contemplated that escrow component 350 may be configured to provide pet owners and prospective pet adopters with a data room in which any of various due diligence items from either the prospective pet adopter (e.g., video of backyard) or the pet owner (e.g., proof of vaccinations) may be uploaded and evaluated. Once all the conditions of escrow are met, it is contemplated that escrow component 350 may be configured to facilitate a transfer of funds, if any, from the prospective pet adopter to the pet owner and/or vice versa.

### Exemplary Pet-Matching Methodology

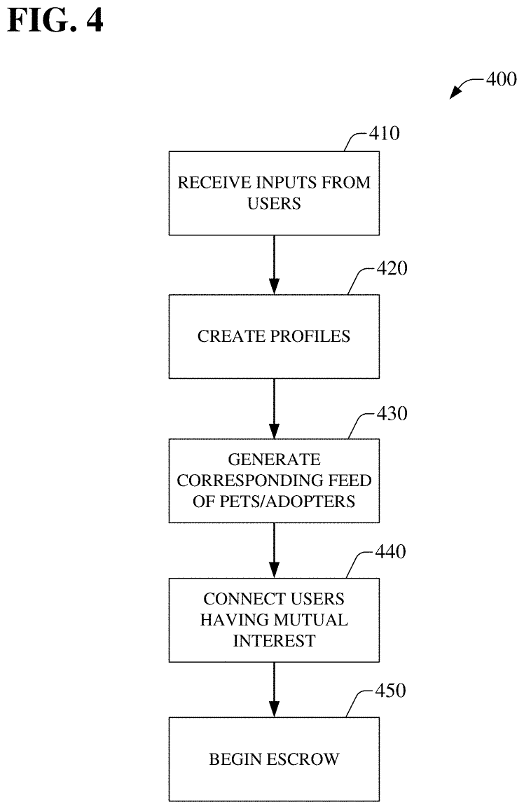

Referring next to FIG. 4, a flow chart illustrating an exemplary method that facilitates matching pet owners with prospective pet adopters according to an embodiment is provided. As illustrated, process 400 includes a series of acts that may be performed by a management system that includes at least one computing device (e.g., management system 300) according to an aspect of the subject specification. For instance, process 400 may be implemented by employing a processor to execute computer executable instructions stored on a computer readable storage medium to implement the series of acts. In another embodiment, a computer-readable storage medium comprising code for causing at least one computer to implement the acts of process 400 is contemplated.

In an aspect, process 400 begins at act 410 with the management system 300 receiving inputs from users, which are then used to generate corresponding profiles (e.g., owner profiles 232, adopter profiles 234, and/or pet profiles 236) at act 420. Process 400 then proceeds to act 430 where the management system 300 provides pet adopters with a feed of pet profiles that meet the pet adopters' search criteria, and provides pet owners with a feed of adopter profiles that meet the pet owners' search criteria. Once both a pet owner and a prospective pet adopter indicate mutual preliminary interest, management system 300 connects them at act 440 so that they can determine whether to open escrow. If so, process 400 then concludes at act 450 with the opening of escrow.

### Exemplary Networked and Distributed Environments

One of ordinary skill in the art can appreciate that various embodiments for implementing the use of a computing device and related embodiments described herein can be implemented in connection with any computer or other client or server device, which can be deployed as part of a computer network or in a distributed computing environment, and can be connected to any kind of data store. Moreover, one of ordinary skill in the art will appreciate that such embodiments can be implemented in any computer system or environment having any number of memory or storage units, and any number of applications and processes occurring across any number of storage units. This includes, but is not limited to, an environment with server computers and client computers deployed in a network environment or a distributed computing environment, having remote or local storage.

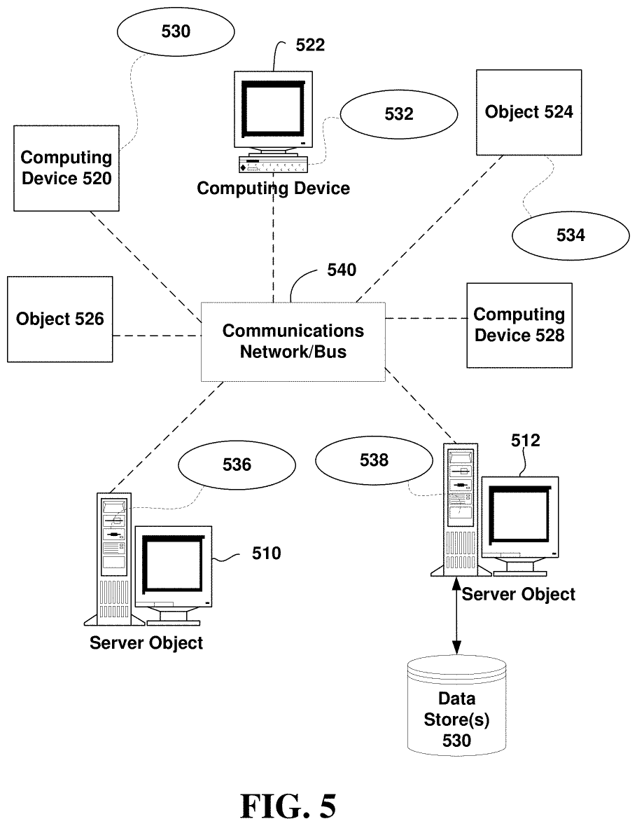

FIG. 5 provides a non-limiting schematic diagram of an exemplary networked or distributed computing environment. The distributed computing environment comprises computing objects or devices 510, 512, etc. and computing objects or devices 520, 522, 524, 526, 528, etc., which may include programs, methods, data stores, programmable logic, etc., as represented by applications 530, 532, 534, 536, 538. It can be appreciated that computing objects or devices 510, 512, etc. and computing objects or devices 520, 522, 524, 526, 528, etc. may comprise different devices, such as PDAs (personal digital assistants), audio/video devices, mobile phones, MP3 players, laptops, etc.

Each computing object or device 510, 512, etc. and computing objects or devices 520, 522, 524, 526, 528, etc. can communicate with one or more other computing objects or devices 510, 512, etc. and computing objects or devices 520, 522, 524, 526, 528, etc. by way of the communications network 540, either directly or indirectly. Even though illustrated as a single element in FIG. 5, network 540 may comprise other computing objects and computing devices that provide services to the system of FIG. 5, and/or may represent multiple interconnected networks, which are not shown. Each computing object or device 510, 512, etc. or 520, 522, 524, 526, 528, etc. can also contain an application, such as applications 530, 532, 534, 536, 538, that might make use of an API (application programming interface), or other object, software, firmware and/or hardware, suitable for communication with or implementation of the disclosed aspects in accordance with various embodiments.

There are a variety of systems, components, and network configurations that support distributed computing environments. For example, computing systems can be connected together by wired or wireless systems, by local networks or widely distributed networks. Currently, many networks are coupled to the Internet, which provides an infrastructure for widely distributed computing and encompasses many different networks, though any network infrastructure can be used for exemplary communications made incident to the techniques as described in various embodiments.

Thus, a host of network topologies and network infrastructures, such as client/server, peer-to-peer, or hybrid architectures, can be utilized. In a client/server architecture, particularly a networked system, a client is usually a computer that accesses shared network resources provided by another computer, e.g., a server. In the illustration of FIG. 5, as a non-limiting example, computing objects or devices 520, 522, 524, 526, 528, etc. can be thought of as clients and computing objects or devices 510, 512, etc. can be thought of as servers where computing objects or devices 510, 512, etc. provide data services, such as receiving data from computing objects or devices 520, 522, 524, 526, 528, etc., storing of data, processing of data, transmitting data to computing objects or devices 520, 522, 524, 526, 528, etc., although any computer can be considered a client, a server, or both, depending on the circumstances. Any of these computing devices may be processing data, or requesting services or tasks that may implicate aspects and related techniques as described herein for one or more embodiments.

A server is typically a remote computer system accessible over a remote or local network, such as the Internet or wireless network infrastructures. The client process may be active in a first computer system, and the server process may be active in a second computer system, communicating with one another over a communications medium, thus providing distributed functionality and allowing multiple clients to take advantage of the information-gathering capabilities of the server. Any software objects utilized pursuant to the user profiling can be provided standalone, or distributed across multiple computing devices or objects.

In a network environment in which the communications network/bus 540 is the Internet, for example, the computing objects or devices 510, 512, etc. can be Web servers with which the computing objects or devices 520, 522, 524, 526, 528, etc. communicate via any of a number of known protocols, such as HTTP. As mentioned, computing objects or devices 510, 512, etc. may also serve as computing objects or devices 520, 522, 524, 526, 528, etc., or vice versa, as may be characteristic of a distributed computing environment.

### Exemplary Computing Device

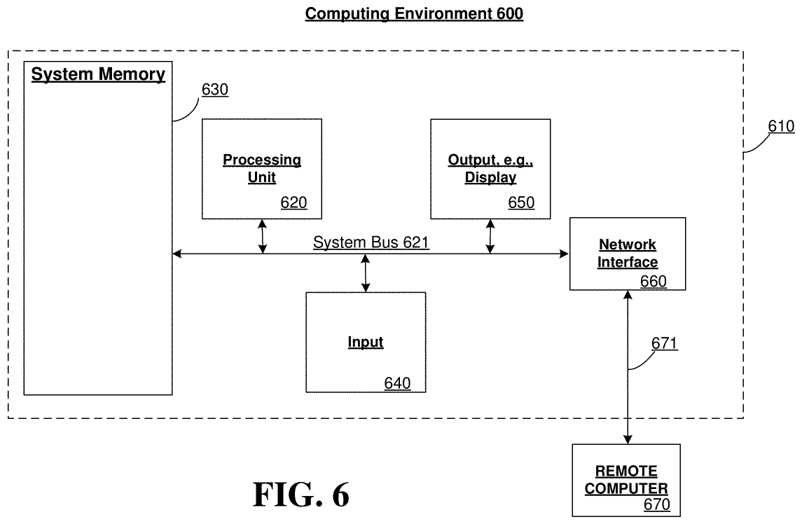

As mentioned, several of the aforementioned embodiments apply to any device wherein it may be desirable to include a computing device to facilitate implementing the aspects disclosed herein. It is understood, therefore, that handheld, portable and other computing devices and computing objects of all kinds are contemplated for use in connection with the various embodiments described herein. Accordingly, the below general purpose remote computer described below in FIG. 6 is but one example, and the embodiments of the subject disclosure may be implemented with any client having network/bus interoperability and interaction.

Although not required, any of the embodiments can partly be implemented via an operating system, for use by a developer of services for a device or object, and/or included within application software that operates in connection with the operable component(s). Software may be described in the general context of computer executable instructions, such as program modules, being executed by one or more computers, such as client workstations, servers or other devices. Those skilled in the art will appreciate that network interactions may be practiced with a variety of computer system configurations and protocols.

FIG. 6 thus illustrates an example of a suitable computing system environment 600 in which one or more of the embodiments may be implemented, although as made clear above, the computing system environment 600 is only one example of a suitable computing environment and is not intended to suggest any limitation as to the scope of use or functionality of any of the embodiments. The computing environment 600 is not to be interpreted as having any dependency or requirement relating to any one or combination of components illustrated in the exemplary operating environment 600.

With reference to FIG. 6, an exemplary remote device for implementing one or more embodiments herein can include a general purpose computing device in the form of a handheld computer 610. Components of handheld computer 610 may include, but are not limited to, a processing unit 620, a system memory 630, and a system bus 621 that couples various system components including the system memory to the processing unit 620.

Computer 610 typically includes a variety of computer readable media and can be any available media that can be accessed by computer 610. The system memory 630 may include computer storage media in the form of volatile and/or nonvolatile memory such as read only memory (ROM) and/or random access memory (RAM). By way of example, and not limitation, memory 630 may also include an operating system, application programs, other program modules, and program data.

A user may enter commands and information into the computer 610 through input devices 640 A monitor or other type of display device is also connected to the system bus 621 via an interface, such as output interface 650. In addition to a monitor, computers may also include other peripheral output devices such as speakers and a printer, which may be connected through output interface 650.

The computer 610 may operate in a networked or distributed environment using logical connections to one or more other remote computers, such as remote computer 670. The remote computer 670 may be a personal computer, a server, a router, a network PC, a peer device or other common network node, or any other remote media consumption or transmission device, and may include any or all of the elements described above relative to the computer 610. The logical connections depicted in FIG. 6 include a network 671, such local area network (LAN) or a wide area network (WAN), but may also include other networks/buses. Such networking environments are commonplace in homes, offices, enterprise-wide computer networks, intranets and the Internet.

As mentioned above, while exemplary embodiments have been described in connection with various computing devices, networks and advertising architectures, the underlying concepts may be applied to any network system and any computing device or system in which it is desirable to implement the aspects disclosed herein.

There are multiple ways of implementing one or more of the embodiments described herein, e.g., an appropriate API, tool kit, driver code, operating system, control, standalone or downloadable software object, etc. which enables applications to implement the aspects disclosed herein. Embodiments may be contemplated from the standpoint of an API (or other software object), as well as from a software or hardware object that facilitates implementing the aspects disclosed herein in accordance with one or more of the described embodiments. Various implementations and embodiments described herein may have aspects that are wholly in hardware, partly in hardware and partly in software, as well as in software.

The word “exemplary” is used herein to mean serving as an example, instance, or illustration. For the avoidance of doubt, the subject matter disclosed herein is not limited by such examples. In addition, any aspect or design described herein as “exemplary” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, for the avoidance of doubt, such terms are intended to be inclusive in a manner similar to the term “comprising” as an open transition word without precluding any additional or other elements.

As mentioned, the various techniques described herein may be implemented in connection with hardware or software or, where appropriate, with a combination of both. As used herein, the terms “component,” “system” and the like are likewise intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on computer and the computer can be a component. One or more components may reside within a process and/or thread of execution and a component may be localized on one computer and/or distributed between two or more computers.

The aforementioned systems have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it is noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but generally known by those of skill in the art.

In view of the exemplary systems described supra, methodologies that may be implemented in accordance with the disclosed subject matter can be appreciated with reference to the flowcharts of the various figures. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Where non-sequential, or branched, flow is illustrated via flowchart, it can be appreciated that various other branches, flow paths, and orders of the blocks, may be implemented which achieve the same or a similar result. Moreover, not all illustrated blocks may be required to implement the methodologies described hereinafter.

While in some embodiments, a client side perspective is illustrated, it is to be understood for the avoidance of doubt that a corresponding server perspective exists, or vice versa. Similarly, where a method is practiced, a corresponding device can be provided having storage and at least one processor configured to practice that method via one or more components.

While the various embodiments have been described in connection with the preferred embodiments of the various figures, it is to be understood that other similar embodiments may be used or modifications and additions may be made to the described embodiment for performing the same function without deviating there from. Still further, one or more aspects of the above described embodiments may be implemented in or across a plurality of processing chips or devices, and storage may similarly be affected across a plurality of devices. Therefore, the present invention should not be limited to any single embodiment.

What is claimed is:

1. A method comprising:
generating a plurality of profiles, wherein the plurality of profiles includes a plurality of pet owner profiles, a plurality of pet adopter profiles, and a plurality of pet profiles;
matching at least a first one of the plurality of profiles with at least a second one of the plurality of profiles, wherein the matching is based on criteria included the first one of the plurality of profiles and the second one of the plurality of profiles; and
facilitating an escrow process between a first user corresponding to the first one of the plurality of profiles and a second user corresponding to the second one of the plurality of profiles.

2. The method of claim 1, wherein the matching comprises displaying a subset of the plurality of profiles to the first user via a graphical user interface (GUI).

3. The method of claim 2, wherein the first user is an account holder of one of the plurality of pet owner profiles and the second user is an account holder of one of the plurality of pet adopter profiles, and wherein the matching is initiated by the first user in response to seeing a profile of the second user displayed via the GUI.

4. The method of claim 2, wherein the first user is an account holder of one of the plurality of pet adopter profiles and the second user is an account holder of one of the plurality of pet owner profiles, and wherein the matching is initiated by the first user in response to seeing a profile associated with the second user displayed via the GUI or one of the plurality of pet profiles displayed via the GUI.

5. The method of claim 2, wherein the displaying comprises displaying the subset of the plurality of profiles in a scrollable sequence.

6. The method of claim 5, wherein the matching comprises determining a swipe direction of the first user when scrolling through the scrollable sequence, and wherein the swipe direction determines whether to initiate a potential match.

7. The method of claim 1, wherein the matching comprises anonymizing at least one of an identity of the first user from the second user or an identity of the second user from the first user.

8. The method of claim 1, wherein the matching comprises connecting at least one of the first user or the second user to external resources associated with a listed attribute of the first user or the second user.

9. The method of claim 1, wherein the facilitating comprises vetting at least one listed attribute of the first user or the second user via an external resource, and wherein the escrow is closed upon a completion of the vetting.

10. The method of claim 9, wherein the vetting comprises confirming medical records of a pet associated with the matching, and wherein the confirming comprises obtaining confirmation from the external resource.

11. A system, comprising:
a communication component configured to generate a plurality of profiles, wherein the plurality of profiles includes a plurality of pet owner profiles, a plurality of pet adopter profiles, and a plurality of pet profiles;
a match component configured to match at least a first one of the plurality of profiles with at least a second one of the plurality of profiles, wherein the matching is based on criteria included the first one of the plurality of profiles and the second one of the plurality of profiles; and
an escrow component configured to facilitate an escrow process between a first user corresponding to the first one of the plurality of profiles and a second user corresponding to the second one of the plurality of profiles.

12. A computer-readable storage medium, comprising:
a memory component configured to store computer-readable instructions, the computer-readable instructions including instructions for performing the following acts:
generating a plurality of profiles, wherein the plurality of profiles includes a plurality of pet owner profiles, a plurality of pet adopter profiles, and a plurality of pet profiles;
matching at least a first one of the plurality of profiles with at least a second one of the plurality of profiles, wherein the matching is based on criteria included the first one of the plurality of profiles and the second one of the plurality of profiles; and
facilitating an escrow process between a first user corresponding to the first one of the plurality of profiles and a second user corresponding to the second one of the plurality of profiles.
